(19) United States
(12) Patent Application Publication
(10) Pub. No.: US 2009/0069291 A1
(43) Pub. Date: Mar. 12, 2009

(54) Salt Forms

(75) Inventors: Alan S. Kirschner, Wilmington, DE (US); James A. Osborn, Wilmington, DE (US); James Hulsizer, Wilmington, DE (US)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 12/093,952

(22) PCT Filed: Nov. 18, 2006
(86) PCT No.: PCT/US2006/061069

§ 371 Date: Oct. 23, 2008

Related U.S. Application Data
(60) Provisional application No. 60/737,861, filed on Nov. 18, 2005.

(51) Int. Cl. A61K 31/554; C07D 267/02; A61P 43/00

(52) U.S. Cl. 514/211.13; 540/551

(57) ABSTRACT

The present invention is directed to salts the pharmaceutical compound 11-piperazin-1-yldibenzo[b,f][1,4]thiazepine as well as compositions, preparations, and pharmaceutical uses thereof.

### FIELD OF THE INVENTION

The present invention is directed to salts of the pharmaceutical compound 11-piperazin-1-yldibenzo[b,f][1,4]thiazepine, as well as compositions, preparations, and pharmaceutical uses thereof.

### BACKGROUND OF THE INVENTION

A goal of antipsychotic drug development has been to develop agents with increased efficacy and safety along with fewer of the side effects commonly associated with the older antipsychotic medications. Quetiapine fumarate is described in U.S. Pat. No. 4,879,288, which is incorporated herein by reference. Quetiapine fumarate is able to treat both the positive (hallucinations, delusions) and negative symptoms (emotional withdrawal, apathy) of psychosis and is associated with fewer neurological and endocrine related side effects compared to older agents. Quetiapine fumarate has also been associated with a reduction in hostility and aggression. Quetiapine fumarate is associated with fewer side effects such as EPS, acute dystonia, acute dyskinesia, as well as tardive dyskinesia. Quetiapine fumarate has also helped to, enhance patient compliance with treatment, ability to function and overall quality of life, while reducing recidivism. P. Weiden et al., Atypical antipsychotic drugs and long-term outcome in schizophrenia, 11 J. Clin. Psychiatry, 53-60, 57 (1996). Because of quetiapine fumarate's enhanced tolerability profile its use is particularly advantageous in the treatment of patients that are hypersensitive to the adverse effects of antipsychotics (such as elderly patients).

Derivatives of 11-(piperazin-1-yl)dibenzo[b,f][1,4]-thiazepines and related compounds including metabolites of quetiapine were prepared and evaluated in E. Warawa et al. Behavioral approach to nondyskinetic dopamine antagonists: identification of Seroquel, 44, J. Med. Chem., 372-389 (2001). Quetiapine metabolism has been reported in C. L. Devane et al. Clin. Pharmacokinet., 40(7), 509-522 (2001) wherein the structure of 11-piperazin-1-yldibenzo[b,f][1,4]thiazepine (see Formula I below) was shown in FIG. 1. This compound was reported by Schmutz et al. in U.S. Pat. No. 3,539,573. This compound has also been used in processes for preparing quetiapine as reported in U.S. Pat. No. 4,879,288. It has now been found that 11-piperazin-1-yldibenzo[b,f][1,4]thiazepine is a circulating metabolite of quetiapine in humans.

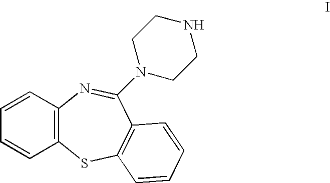

Because pharmaceutically active compounds and compositions having, for example, improved properties over existing forms are consistently sought, there is an ongoing need for improved forms of existing drug molecules and their active, circulating metabolites. The salts of 11-piperazin-1-yldibenzo[b,f][1,4]thiazepine described herein are directed toward this end.

### SUMMARY OF THE INVENTION

The present invention provides salt forms of 11-piperazin-1-yldibenzo[b,f][1,4]thiazepine. In some embodiments, the salt form is crystalline.

The present invention further provides compositions containing a salt form of 11-piperazin-1-yldibenzo[b,f][1,4]thiazepine. In some embodiments, the compositions comprise a pharmaceutically acceptable carrier. In further embodiments, the compositions comprise at least one benzodiazepine, 5-HT_{1A} ligand, 5-HT_{1B} ligand, 5-HT_{1D} ligand, mGluR2A agonist, mGluR5 antagonist, antipsychotic, NK1 receptor antagonist, antidepressant, serotonin reuptake inhibitor or a mood stabilizer.

The present invention further provides processes for preparing the salt forms of 11-piperazin-1-yldibenzo[b,f][1,4]thiazepine.

The present invention further provides the salt forms of 11-piperazin-1-yldibenzo[b,f][1,4]thiazepine prepared by the processes described herein.

The present invention further provides methods of treating at least one symptom or condition associated with but not limited to: 1) Schizophrenia and other Psychotic Disorders including but not limited to Psychotic Disorder, Schizophreniform Disorder, Schizoaffective Disorder, Delusional Disorder, Brief Psychotic Disorder, Shared Psychotic Disorder, and Psychotic Disorder Due to a General Medical Condition; 2) Dementia and other Cognitive Disorders; 3) Anxiety Disorders including but not limited to Panic Disorder Without Agoraphobia, Panic Disorder With Agoraphobia, Agoraphobia Without History of Panic Disorder, Specific Phobia, Social Phobia, Obsessive-Compulsive Disorder, Posttraumatic Stress Disorder, Acute Stress Disorder, Generalized Anxiety Disorder and Generalized Anxiety Disorder Due to a General Medical Condition. 4) Mood Disorders including but not limited to a) Depressive Disorders, including but not limited to Major Depressive Disorder and Dysthymic Disorder and b) Bipolar Depression and/or Bipolar mania including but not limited to Bipolar I Disorder, including but not limited to those with manic, depressive or mixed episodes, and Bipolar II Disorder, c) Cyclothymic Disorder, d) Mood Disorder Due to a General Medical Condition; 5) Sleep Disorders; 6) Disorders Usually First Diagnosed in Infancy, Childhood, or Adolescence including but not limited to Mental Retardation, Learning Disorders, Motor Skills Disorder, Communication Disorders, Pervasive Developmental Disorders, Attention-Deficit and Disruptive Behavior Disorders, Feeding and Eating Disorders of Infancy or Early Childhood, Tic Disorders, and Elimination Disorders; 7) Substance-Related Disorders including but not limited to Substance Dependence, Substance Abuse, Substance Intoxication, Substance Withdrawal, Alcohol-Related Disorders, Amphetamine (or Amphetamine-Like)-Related Disorders, Caffeine-Related Disorders, Cannabis-Related Disorders, Cocaine-Related Disorders, Hallucinogen-Related Disorders, Inhalant-Related Disorders, Nicotine-Related Disorders, Opioid-Related Disorders, Phencyclidine (or Phencyclidine-Like)-Related Disorders, and Sedative-, Hypnotic- or Anxiolytic-Related Disorders; 8) Attention-Deficit and Disruptive Behavior Disorders; 9) Eating Disorders; 10) Personality Disorders including but not limited to Obsessive-Compulsive Personality Disorder; and 11) Impulse-Control Disorders, comprising administering to a mammal a therapeutically effective amount of a salt form of the invention.

The present invention further provides the salts of the invention for use in treating the symptoms or conditions provided herein.

The present invention further provides the salts of the invention for use in the manufacture of a medicament for the treatment of symptoms or conditions provided herein.

### BRIEF DESCRIPTION OF THE DRAWINGS

FIG. 1 shows a TGA/DSC thermogram of the L-tartaric acid salt.

FIG. 2 shows a DVS analysis for the L-tartaric acid salt.

FIG. 3 shows a TGA/DSC thermogram of the fumaric acid salt.

FIG. 4 shows a DVS analysis for the fumaric acid salt.

FIG. 5 shows a TGA/DSC thermogram of the methanesulfonic acid salt.

FIG. 6 shows a DVS analysis for the methanesulfonic acid salt.

FIG. 7 shows a TGA/DSC thermogram of the sulfuric acid salt.

FIG. 8 shows a DVS analysis for the sulfuric acid salt.

FIG. 9 shows a TGA/DSC thermogram of the phosphoric acid salt.

FIG. 10 shows a DVS analysis for the phosphoric acid salt.

### DETAILED DESCRIPTION

The compound of Formula I is a dibenzothiazepine that has shown antidopaminergic activity. It has been shown to interact with a broad range of neurotransmitter receptors but has a higher affinity for serotonin (5-HT₂) receptors relative to dopamine (D₂) receptors in the brain. Preliminary positron emission topography (PET) scans of primate subjects showed that the compound of Formula I reaches the brain and occupies D1, D₂, 5-HT_{2A}, and 5-HT_{1A} receptors and the 5HT Transporter. However, the compound of Formula I was not shown to be efficacious in a mouse standard apomorphine swim test (p.o.) and in a rat D-Amphetamine locomotor activity test (s.c.).

The compound of Formula I has also been shown to have partial 5HT_{1A} agonist activity and has shown in-vivo efficacy in mouse and rat models for depression. The compound of Formula I may be used as an antipsychotic with a reduction in the potential to cause side effects such as acute dystonia, acute dyskinesia, as well as tardive dyskinesia typically seen with antipsychotics. Results generated from alpha receptor binding data further suggest that the compound of Formula I will have improved tolerability over that of quetiapine and suggest that one would observe a reduced incidence of hypotension. Further the compound of Formula I may be used to treat patients of all ages and is advantageous in the treatment of elderly patients.

The present invention provides, inter alia, salt forms of the pharmaceutical compound II′-piperazin-1-yldibenzo[b,f][1,4]thiazepine useful, for example, as an antipsychotic. Exemplary salt forms of the invention include:

a) L-tartaric acid;

b) fumaric acid;

c) methanesulfonic acid;

d) sulfuric acid,

e) phosphoric acid;

f) acetic acid;

g) L-ascorbic acid;

h) benzoic acid;

i) benzene sulfonic acid

j) 2-(benzoylamino)acetic acid;

k) (+)-(1S)-camphor-10-sulfonic acid;

1) citric acid;

m) 1,2-ethanedisulfonic acid;

n) ethanesulfonic acid;

o) formic acid;

p) gluconic acid;

q) lactic acid;

r) maleic acid;

s) L-malic acid;

t) DL-mandelic acid;

u) naphthalene-1,5-disulfonic acid;

v) naphthalene-2-sulfonic acid;

w) nitric acid;

x) octadecanoic acid;

y) p-toluenesulfonic acid;

z) propanedioic acid;

aa) succinic acid;

bb) undec-10-enoic acid; or

cc) p-xyelene-2-sulfonic acid salts of 11-piperazin-1-yldibenzo[b,f][1,4]thiazepine.

Advantages of these salts are numerous and include their propensity to form tractable solids, including crystalline solids, thereby facilitating preparation, purification, formulation, and administration of the drug.

The salts of the invention can take on any solid form including amorphous or crystalline forms, as well as mixtures thereof. In some embodiments, the salts are crystalline. Water or solvent molecules can also be contained within the salts forming hydrates and/or solvates. Alternatively, the salts can be anhydrous or non-solvated. The salts of the invention are further characterized by having one or more acid molecules per molecule of 11-piperazin-1-yldibenzo[b,f][1,4]thiazepine. In some embodiments, the salt contains about one molecule of acid to every one molecule of 11-piperazin-1-yldibenzo[b,f][1,4]thiazepine (monoacid). In further embodiments, the salt contains more than one molecule of acid to every one molecule of 11-piperazin-1-yldibenzo[b,f][1,4]thiazepine. In yet further embodiments, the salt contains about two molecules of acid to every one molecule of 11-piperazin-1-yldibenzo[b,f][1,4]thiazepine (diacid).

Methods of characterizing salts are routine in the art and include numerous solid state methods such as X-ray powder diffraction (XRPD), differential scanning calorimetry (DSC), Raman scattering, and solid state nuclear magnetic resonance (NMR). Methods for detecting hydration or solvation (or the absence thereof) include thermogravimetric analysis (TGA), dynamic vapor sorption (DVS), Karl Fisher titrations, elemental analyses, and the like.

The salt forms of the invention can be prepared by any suitable manner according to routine methods in the art. For example, 11-piperazin-1-yldibenzo[b,f][1,4]thiazepine can be combined together with an appropriate acid (e.g., L-tartaric acid, fumaric acid, methanesulfonic acid, sulfuric acid, phosphoric acid, etc.) in a solvent followed by precipitating the resulting salt from the solvent. The molar ratio of free base to acid can vary and can be, for example, about 5:1 to about 1:5, about 3:1 to about 1:3, about 1:1 to about 1:3, or about 1:1 to about 1:2. In some embodiments, the molar ratio of free base to acid is about 1:1. In some embodiments, the molar ratio of free base to acid is about 1:2. In some embodiments, the acid is provided in molar excess relative to free base. Suitable solvents include any solvent in which 11-piperazin-1-yldibenzo[b,f][1,4]thiazepine is at least partially soluble. Such solvents include polar organic solvents such as acetonitrile, acetone, methanol, ethyl acetate, mixtures thereof and the like. Other suitable solvents include, dimethylsulfoxide, dimethylformamide, dichlormethane, ethanol, isopropanol, and the like. Suitable solvents for the preparation of hydrated salt forms can further include water or water/organic solvent mixtures. In some embodiments, the solvent is acetonitrile, methanol, acetone, ethyl acetate or a mixture thereof.

Precipitation of the salt forms of the invention can be carried out by any suitable manner according to routine methods. For example, solutions of salts of 11-piperazin-1-yldibenzo[b,f][1,4]thiazepine can be evaporated, cooled, treated with antisolvent, or combinations thereof. Treatment with antisolvent can be carried out by layering or vapor diffusion techniques. Suitable antisolvents include weakly polar organic solvents that are miscible with the crystallizing solvent such as ethers (e.g., diethyl ether, THF, methyl-t-butyl ether, and the like) and hydrocarbons (e.g., pentane, hexanes, cyclohexane, benzene, toluene, and the like).

The salt forms of the invention can be provided in the form of a composition containing the salt form (in any detectable amount) with at least one other substance. For example, a composition can contain at least about 10, at least about 20, at least about 30, at least about 40, at least about 50, at least about 60, at least about 70, at least about 80, at least about 90, at least about 95, at least about 96, at least about 97, at least about 98, at least about 99% by weight of a salt form of the invention.

In some embodiments, the composition is a pharmaceutical composition which includes a salt of the invention in combination with a pharmaceutically acceptable carrier. In some embodiments, the pharmaceutical composition includes up to about 750 mg of a salt of the invention, particularly in an amount from about 75 mg to about 750 mg. In another embodiment, the pharmaceutical composition comprises from about 1 mg to about 600 mg of a salt of the invention. In a further embodiment, the pharmaceutical composition comprises from about 100 mg to about 400 mg of a salt of the invention.

In further embodiments, the pharmaceutical composition includes the salt of the invention in combination with a pharmaceutically acceptable carrier and at least one further active ingredient. Example further active ingredients include benzodiazepines, 5-HT_{1A} ligands, 5-HT_{1B} ligands, 5-HT_{1D} ligands, mGluR2A agonists, mGluR5 antagonists, antipsychotics, NK1 receptor antagonists, antidepressants, or serotonin reuptake inhibitors.

The pharmaceutical compositions of the invention can accordingly be obtained by conventional procedures using conventional pharmaceutical excipients. In malting the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. Pharmaceutical compositions intended for oral use can further contain, for example, one or more coloring, sweetening, flavoring and/or preservative agents.

The composition of the invention can be administered by any route including orally, intramuscularly, subcutaneously, topically, intranasally, intraperitoneally, intrathoracially, intravenously, epidurally, intrathecally, intracerebroventricularly and by injection into the joints.

The amount of active ingredient that is combined with one or more excipients to produce a single dosage form will necessarily vary depending upon the host treated and the particular route of administration. The size of the dose for therapeutic or prophylactic purposes of the active compound(s) will naturally vary according to the nature and severity of the symptoms or conditions, the age and sex of the animal or patient and the route of administration, according to well known principles of medicine.

The present invention further provides methods of treating at least one symptom or condition associated with schizophrenia and other psychotic disorders (e.g., psychotic disorder, psychosis); dementia and other cognitive disorders, anxiety disorders (e.g., generalized anxiety disorder); mood disorders (e.g., depressive disorders, major depressive disorders; bipolar disorders including bipolar I and II, bipolar mania, bipolar depression); sleep disorders; disorders usually first diagnosed in infancy, childhood, or adolescence (e.g., attention-deficit disorder and disruptive behavior disorders); and neurodegenerative disorders comprising administering to a mammal a pharmaceutically effective amount of a salt form of the invention or composition containing one or more of the same. In some embodiments, the symptoms and conditions include but are not limited to anxiety, agitation, hostility, panic, eating disorders, affective symptoms, mood symptoms, negative and positive psychotic symptoms commonly associated with psychosis and neurodegenerative disorders. In some embodiments, the symptoms and conditions are any of psychosis, schizophrenia, bipolar I, and anxiety.

In some embodiments, the present invention further provides methods of treating at least one symptom or condition associated with but not limited to: 1) Schizophrenia and other Psychotic Disorders including but not limited to Psychotic Disorder, Schizophreniform Disorder, Schizoaffective Disorder, Delusional Disorder, Brief Psychotic Disorder, Shared Psychotic Disorder, and Psychotic Disorder Due to a General Medical Condition; 2) Dementia and other Cognitive Disorders; 3) Anxiety Disorders including but not limited to Panic Disorder Without Agoraphobia, Panic Disorder With Agoraphobia, Agoraphobia Without History of Panic Disorder, Specific Phobia, Social Phobia, Obsessive-Compulsive Disorder, Posttraumatic Stress Disorder, Acute Stress Disorder, Generalized Anxiety Disorder and Generalized Anxiety Disorder Due to a General Medical Condition; 4) Mood Disorders including but not limited to a) Depressive Disorders, including but not limited to Major Depressive Disorder and Dysthymic Disorder and b) Bipolar Depression and/or Bipolar mania including but not limited to Bipolar I Disorder, including but not limited to those with manic, depressive or mixed episodes, and Bipolar II Disorder, c) Cyclothymic Disorder, d) Mood Disorder Due to a General Medical Condition; 5) Sleep Disorders; 6) Disorders Usually First Diagnosed in Infancy, Childhood, or Adolescence including but not limited to Mental Retardation, Learning Disorders, Motor Skills Disorder, Communication Disorders, Pervasive Developmental Disorders, Attention-Deficit and Disruptive Behavior Disorders, Feeding and Eating Disorders of Infancy or Early Childhood, Tic Disorders, and Elimination Disorders; 7) Substance-Related Disorders including but not limited to Substance Dependence, Substance Abuse, Substance Intoxication, Substance Withdrawal, Alcohol-Related Disorders, Amphetamine (or Amphetamine-Like)-Related Disorders, Caffeine-Related Disorders, Cannabis-Related Disorders, Cocaine-Related Disorders, Hallucinogen-Related Disorders, Inhalant-Related Disorders, Nicotine-Related Disorders, Opioid-Related Disorders, Phencyclidine (or Phencyclidine-Like)-Related Disorders, and Sedative-, Hypnotic- or Anxiolytic-Related Disorders; 8) Attention-Deficit and Disruptive Behavior Disorders; 9) Eating Disorders; 10) Personality Disorders including but not limited to Obsessive-Compulsive Personality Disorder; and 11) Impulse-Control Disorders.

The above conditions and disorders are defined for example in the American Psychiatric Association Diagnostic and Statistical Manual of Mental Disorders, Fourth Edition, Text Revision, Washington, D.C., American Psychiatric Association, 2000. Substance abuse and substance dependence and related disorders are also defined therein. This Manual may also be referred to for greater detail on the symptoms and diagnostic features associated with substance use, abuse and dependence. Typical substances that lead to substance abuse and substance dependence include drugs such as amphetamines, cannabis, cocaine, crack, hallucinogenic agents, inhalants, opioids, phencyclidine, sedatives, hypnotics, anxiolytic agents and alcohol. Nicotine can also lead to substance dependence.

The present invention further provides methods of treating at least one symptom or condition described herein by administering to a mammal a pharmaceutically effective amount of the salt of the invention, or composition containing one or more of the same, and a therapeutically effective amount of at least one other therapeutically active agent selected from benzodiazepines, 5-HT_{1A} ligands, 5-HT_{1B} ligands, 5-HT_{1D} ligands, mGluR2A agonists, mGluR5 antagonists, antipsychotics, NK1 receptor antagonists, antidepressants, serotonin reuptake inhibitors, and mood stabilizers.

Exemplary benzodiazepines include but are not limited to adinazolam, alprazolam, bromazepam, clonazepam, chlorazepate, chlordiazepoxide, diazepam, estazolam, flurazepam, balezepam, lorazepam, midazolam, nitrazepam, oxazepam, quazepam, temazepam, triazolam and equivalents thereof.

Exemplary 5-HT_{1A} and/or 5HT_{1B} ligands include but are not limited to buspirone, alnespirone, elzasonan, ipsapirone, gepirone, zopiclone and equivalents thereof.

Exemplary mGluR2 agonists may include (1S,3R)-1-aminocyclopentane-1,3-dicarboxylic acid, (2S,3S,4S)alpha-(carboxycyclopropyl)glycine, and 3,5-dihydroxyphenylglycine.

Exemplary antidepressants include but are not limited to maprotiline, amitriptyline, clomipramine, desipramine, doxepin, imipramine, nortryptyline, protriptyline, trirnipramine, SSRIs and SNRIs such as fluoxetine, paroxetine, citalopram, escitalopram, sertraline, venlafaxine, fluoxamine, and reboxetine.

Exemplary antipsychotics include but are not limited to clozapine, risperidone, quetiapine, olanzapine, amisulpride, sulpiride, zotepine, chlorpromazine, haloperidol, ziprasidone, and sertindole.

Exemplary mood stabilizers may include but are not limited to Valproic acid (valproate) and its derivative (e.g. divalproex), lamotrigine, lithium, verapamil, carbamazepine and gabapentin.

Administration of two or more active agents can be carried out in combination, e.g., as part of the same pharmaceutical composition, or separately (e.g., serially or consecutively) as part of an appropriate dose regimen designed to obtain the benefits of combination therapy. The appropriate dose regimen, the amount of each dose of an active agent administered, and the specific intervals between doses of each active agent will depend upon the subject being treated, the specific active agent being administered and the nature and severity of the specific disorder or condition being treated.

In general, the salts of this invention, when used as either a single active agent or when used in combination with another active agent, will be administered to a mammal in an amount up to about 750 mg per day in single or divided doses, particularly from about 75 mg to about 750 mg per day. In another aspect of the invention the amount of the salts may be administered from about 1 mg to about 600 mg per day. In another aspect of the invention, the salts may be administered in amount from about 100 mg to about 400 mg per day. Such compounds may be administered on a regimen of up to 6 times per day, preferably 1 to 4 times per day. Variations can occur depending upon the mammal being treated and the individual response to the treatment, as well as on the type of pharmaceutical formulation chosen and the time period and interval at which such administration is carried out. In some instances, dosage levels below the lower limit of the aforesaid range may be more than adequate, while in other cases larger doses may be employed to achieve the desired effect, provided that such larger doses are first divided into several small doses for administration throughout the day.

In some embodiments, the salt form is administered comprising a predetermined dosage to a mammal between one and four times a day, wherein the predetermined dosage is from about 1 mg to about 600 mg.

The present invention also provides a method of treating the symptoms or conditions provided herein comprising the step of administering an initial predetermined dosage of the salt form to a human patient twice a day, wherein the predetermined dosage is between 1 mg and 30 mg with increases in increments of 1-50 mg twice daily on the second and third day as tolerated. Thereafter, further dosage adjustments can be made at intervals of 2 days or greater.

A clinician may determine the effective amount by using numerous methods already known in the art. The term “treating” within the context of the present invention encompasses to administer an effective amount of a salt described herein to mitigate either a pre-existing disease state, acute or chronic, or a recurring symptom or condition. This definition also encompasses prophylactic therapies for prevention of recurring conditions and continued therapy for chronic disorders.

The term “treating” within the context of the present invention is meant to encompass the administration of a therapeutically effective amount of the crystalline form of the present invention to mitigate or inhibit either a pre-existing disease state, acute or chronic, or a recurring symptom or condition. Also encompassed are prophylactic therapies for prevention of recurring conditions and continued therapy for chronic disorders.

The term “mammal” is meant to refer to any warm-blooded animal, preferably a human. In some embodiments, the mammal is in need of treatment because it is suffering from or prone to developing one or more of the symptoms, diseases or disorders described above.

Any or all of the salt forms described herein, including any combination thereof, can be used in the preparation of a medicament for the treatment of any of the diseases, disorders, or conditions described herein.

In order that the invention disclosed herein may be more efficiently understood, examples are provided below. It should be understood that these examples are for illustrative purposes only and are not to be construed as limiting the invention in any manner.

### EXAMPLES

### Example 1

### 11-piperazin-1-yldibenzo[b,f][1,4]thiazepine L-Tartaric acid salt monohydrate

In 5 mL of acetonitrile was dissolved 500 mg (1.693 mmol) of 11-piperazin-1-yldibenzo[b,f][1,4]thiazepine. In 5 mL of acetonitrile was dissolved 254 mg (1.693 mmol) of L-tartaric acid with heating. The solutions were combined and a slightly gummy solid precipitated immediately. The mixture was diluted with an additional 5 mL of acetonitrile. The solid did not dissolve, but triturated into a free-flowing solid. The mixture did not change overnight. The solids were collected, washed with acetonitrile (5 mL), and dried under vacuum at 40° C. resulting in 707 mg crystalline solid (93.8%). mp 170-175° C. (dec). ¹H NMR (DMSO-d₆) was consistent with the title salt.

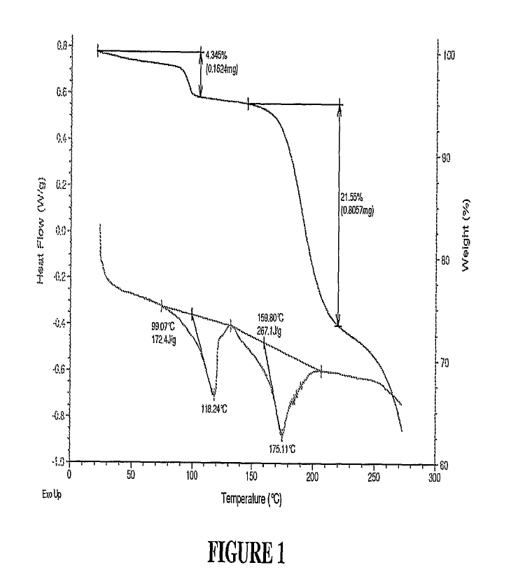

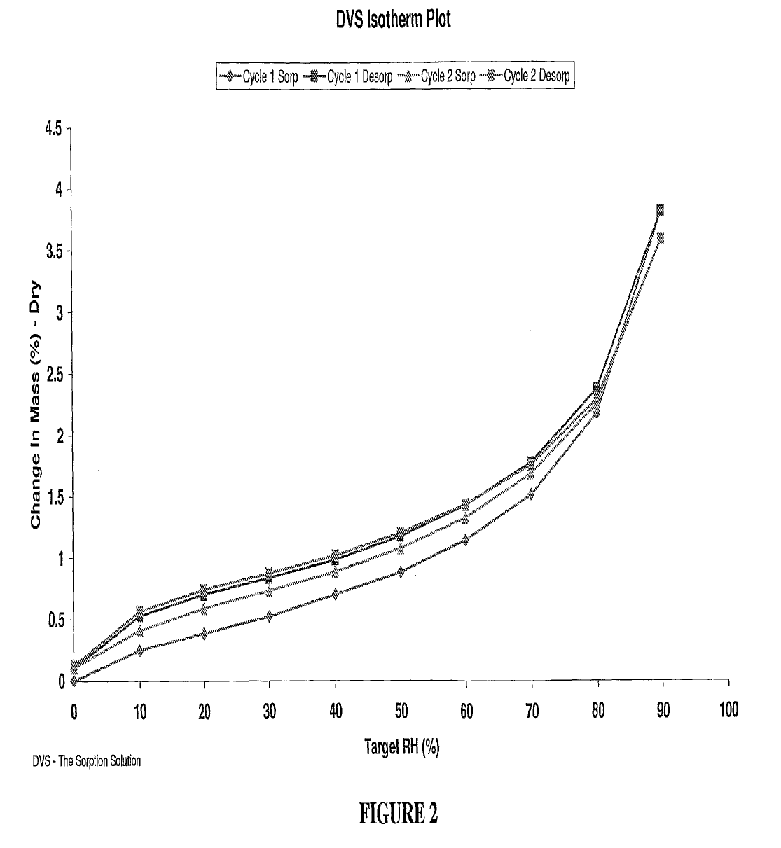

Polarized light microscopy showed that the solid was composed of irregularly shaped crystalline particles. DSC revealed two broad endotherms at 118.3 and 175.1° C. (FIG. 1). The lower temperature endothermic event is likely due to water loss and the higher temperature endothermic event may be due to a melt followed by decomposition. TGA revealed two weight loss transitions (FIG. 1). The lower temperature weight loss transition coincided with the lower temperature DSC event and was measured to correspond to approximately 1 mole equivalent of water. The higher weight loss transition coincided with the higher DSC event. Dynamic vapor sorption (DVS) studies indicated that the salt was hygroscopic (FIG. 2). Moisture gain was reversible with little hysteresis and amounts to about 1 mole equivalent at 90% RH which was in addition to the moisture already contained in the salt.

### Example 2

### 11-piperazin-1-yldibenzo[b,f][1,4]thiazepine fumaric acid salt

In 5 mL of acetonitrile was dissolved 500 mg (1.693 mmol) of 11-piperazin-1-yldibenzo[b,f][1,4]thiazepine. In 5 mL of acetonitrile was dissolved 197 mg fumaric acid (1.693 mmol) with heating. The solutions were combined resulting in precipitation. The solid redissolved upon heating and then crystallized more slowly upon cooling, generating a free-flowing solid. The mixture did not change overnight. The solids were collected, washed with acetonitrile (5 mL), and dried under vacuum at 40° C. resulting in 574 mg (82.3%) of crystalline solid. mp 159-163° C. (dec). ¹H NMR (DMSO-d₆) was consistent with the title salt.

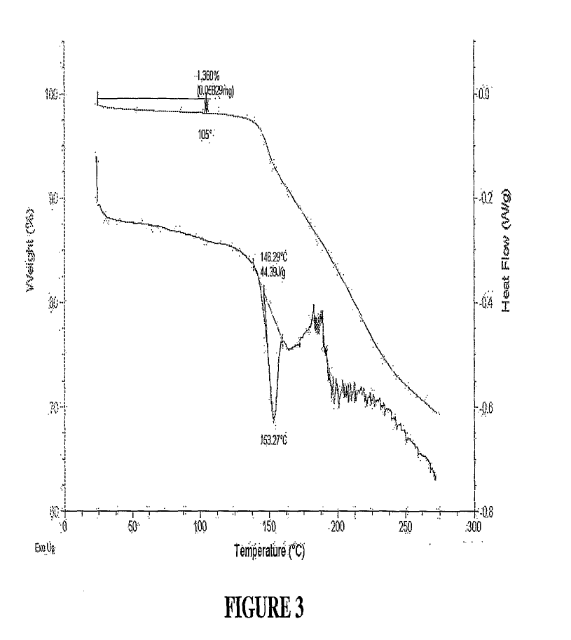

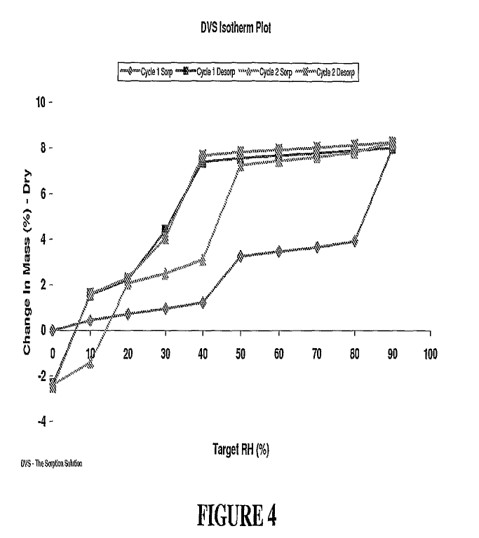

Polarized light microscopy revealed the rod-shaped crystalline particles. DSC revealed one endotherm at 153.3° C. which appeared to be a melt event preceding eventual decomposition at higher temperatures (FIG. 3). TGA revealed 1.4% weight loss in the water/solvent temperature region (FIG. 3). DVS indicated that the salt was hygroscopic with isotherms characteristic of hydrate formation (FIG. 4). The sorption isotherms of each cycle were different, indicating possible form change. The plateau of the first cycle (diamond) between 50 and 80% RH was about equal to 1 mole equivalent of water. The plateau in the same region of the second cycle (triangle) was equal to about 2.5 mole equivalents gained from the starting point of the second cycle at 0% RH. The observation that the second cycle started at a lower mass than the first cycle was probably due to incomplete drying of the sample prior to the cycling.

### Example 3

### 11-piperazin-1-yldibenzo[b,f][1,4]thiazepine methanesulfonic acid salt

In 5 mL of acetonitrile was dissolved 500 mg (1.693 mmol) of 11-piperazin-1-yldibenzo[b,f][1,4]thiazepine. Methanesulfonic acid was dissolved in 5 mL of acetonitrile at room temperature. The solutions were combined resulting in a clear solution. No solid precipitated after several hours at room temperature. Crystals formed upon standing overnight. The solid was broken up, collected, washed with acetonitrile (5 mL), and dried under vacuum at 40° C. resulting in 471 mg (71%) of crystalline material. mp 251-253° C. (sharp). ¹H NMR (DMSO-d₆) was consistent with the title salt.

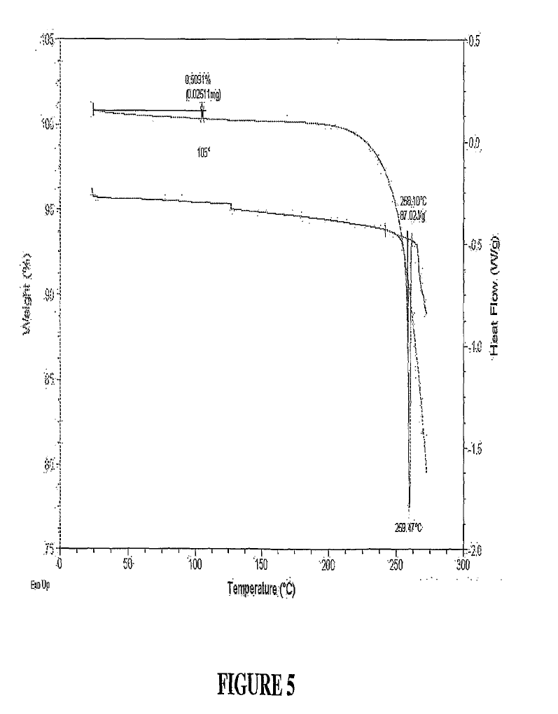

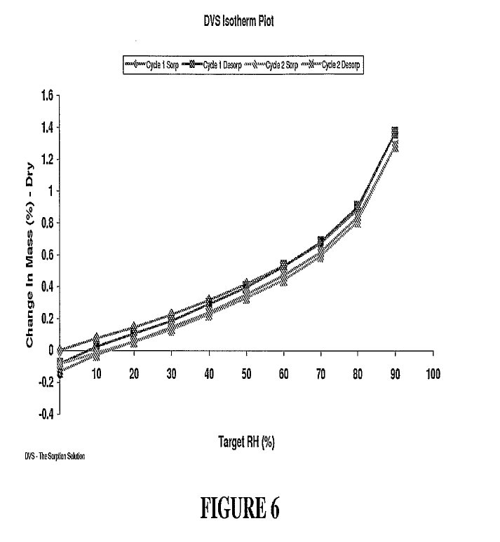

Polarized light microscopy revealed the material to be composed of irregularly shaped crystalline particles. DSC revealed one sharp endothermic event at 259.5° C. (FIG. 5). TGA shows a small weight loss of 0.5% in the water/solvent region (FIG. 5). DVS revealed that the salt is mildly hygroscopic with reversible moisture sorption and no hysteresis (FIG. 6). No evidence of form change was observed.

### Example 4

### 11-piperazin-1-yldibenzo[b,f][1,4]thiazepine sulfuric acid salt

In 5 mL of acetonitrile was dissolved 500 mg (1.693 mmol) of 11-piperazin-1-yldibenzo[b,f][1,4]thiazepine. In 5 mL of acetonitrile at room temperature was dissolved 166 mg (1.693 mmol) H₂SO₄. The solutions were combined resulting in immediate precipitation of a gummy solid. The solid did not redissolve upon reheating, but did triturate into a free-flowing solid. The solids collapsed overnight into a gummy mass which was redissolved in methanol (50 mL) and stripped down to a granular solid which was triturated under acetone (20 mL), filtered, and dried under vacuum at 40° C. resulting in 660 mg (99%). mp 100-105° C. (sharp). ¹H NMR (DMSO-d₆) was consistent with the title salt.

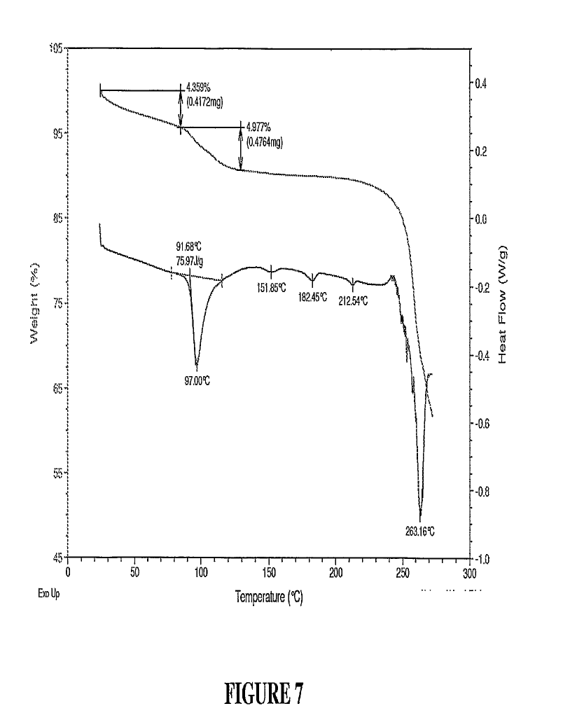

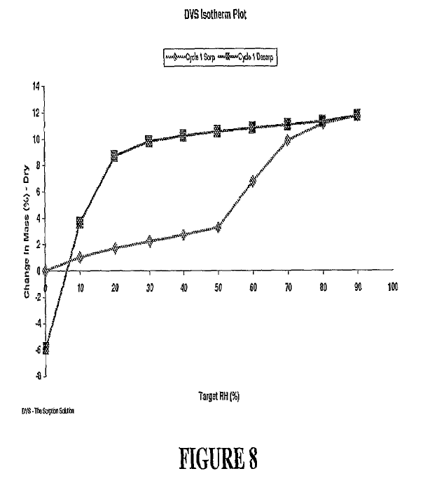

Polarized light microscopy revealed the material to be composed of agglomerates of very small crystalline particles. DSC revealed two large and three small endothermic events (FIG. 7). Of the two large events, the lower temperature event may correspond to water/solvent loss and the higher temperature event may correspond to melt/decomposition. The three small events may correspond to form changes. DVS revealed that the material is hygroscopic with isotherms characteristic of a weak multi-hydrate (FIG. 8). The experiment was terminated near the end of sorption phase of the second cycle. Data from the first cycle indicated that moisture was lost during the desorption phase that had not been lost during the initial drying phase. Approximately 2.5 mole equivalents of water were gained in the first cycle while nearly 4 mole equivalents were gained during the second cycle.

### Example 5

### 11-piperazin-1-yldibenzo[b,f][1,4]thiazepine phosphoric acid salt

In 5 mL of acetonitrile was dissolved 500 mg (1.693 mmol) of 11-piperazin-1-yldibenzo[b,f][1,4]thiazepine. In 5 mL of acetonitrile at room temperature was dissolved H₃PO₄ (85% assay; 163 mg, 1.693 mmol). The solutions were combined resulting in immediate precipitation of a slightly gummy solid. The solid did not redissolve upon reheating, but did triturate into a free-flowing solid. There was no change upon standing overnight. The solids were collected, washed with acetonitrile (5 mL) and dried under vacuum at 40° C. resulting in 588 mg (88.7%) of crystalline solid. mp 227-233° C. (dec). ¹H NMR (DMSO-d₆) was consistent with the title salt.

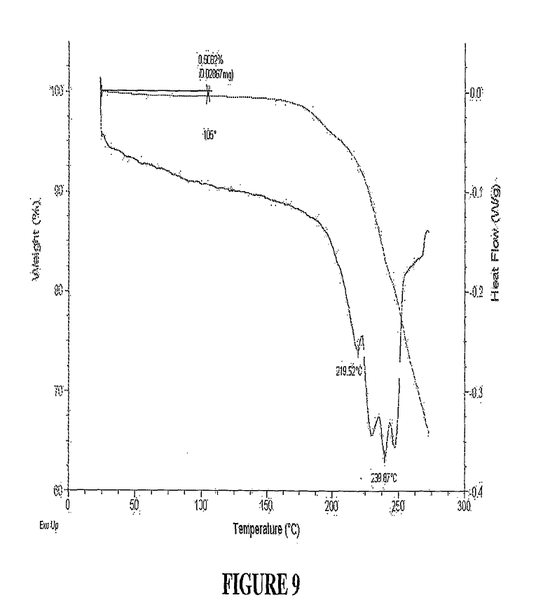

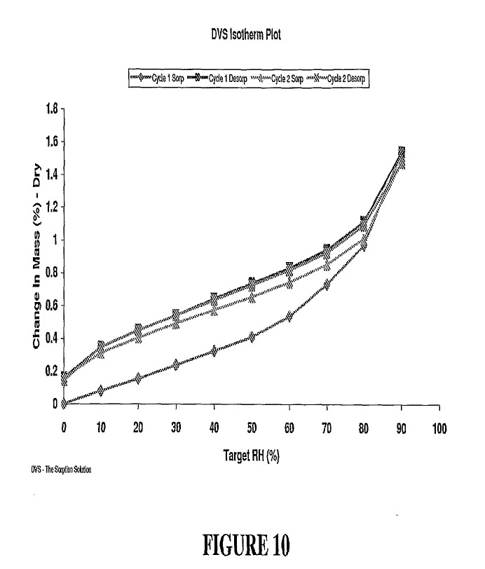

Polarized light microscopy revealed the material to be composed of rod-shaped crystalline particles. DSC revealed a series of endothermic events at higher temperatures likely corresponding to melting and degradation (see FIG. 9). The TGA also indicated slight water loss of 0.5% at 105° C. (see FIG. 9). DVS revealed that the material was slightly hygroscopic (see FIG. 10). The moisture gain was reversible with some hysteresis on the first cycle (characteristic of a channel hydrate). The cycles overlapped well with no evidence of form change.

### Example 6

### Salt screen of 11-piperazin-1-yldibenzo[b,f][1,4]thiazepine

11-piperazin-1-yldibenzo[b,f][1,4] thiazepine was screened for potential salt formation with 30 acids listed below in Table 1. Stock solutions (0.05 M) of each of the acids were prepared in methanol. A stock solution (0.05 M) of amorphous 11-piperazin-1-yldibenzo[b,f][1,4]thiazepine (free base) was also prepared. A 150 μL aliquot of free base solution was mixed with a 150 μL aliquot of each of the acid solutions in individual wells of a glass 96-well plate. Each combination was prepared in duplicate. The methanol was allowed to evaporate either at room temperature (rt) or at 50° C. Each well was then viewed microscopically at a magnification of 40× initially, under crossed-polars, to determine the nature (crystalline or non-crystalline) of any solid material that was formed.

A 300 μL aliquot of another solvent (either acetone, acetonitrile or ethyl acetate) was added to each well. The plate was then sonicated to re-dissolve the solid material, followed by storage at room temperature or 50° C., allowing evaporation of solvent. Each well was then viewed microscopically at a magnification of 40× initially, under crossed-polars, to determine the nature (crystalline or non-crystalline) of any solid material that was formed. Results are shown in Table 1. The symbol “A” indicates formation of solid amorphous particles/film. The symbol “B” indicates formation of amorphous cake. The symbol “C” indicates formation of crystalline particles. The symbol “P” indicates precipitation after mixing and formation of crystalline particles.

TABLE 1

| | Methanol | | Acetonitrile | | Acetone | | Ethyl acetate | |
|---|---|---|---|---|---|---|---|---|
| Acid | RT | 50° C. | RT | 50° C. | RT | 50° C. | RT | 50° C. |
| acetic acid | A | A | A | A | A | A | C | A |
| L-ascorbic acid | A | A | A | A | A | A | A | A |
| benzoic acid | C | A | C | C | C | C | C | C |
| benzene sulfonic acid | A | A | C | C | C | C | C | C |
| 2-(benzyoylamino) | A | A | C | A | C | A | C | C |
| acetic acid | | | | | | | | |
| (+)-(1S)-camphor-10- | A | A | C | A | C | C | C | C |
| sulfonic acid | | | | | | | | |
| citric acid | A | A | A | A | A | A | A | A |
| 1,2-ethanedisulfonic acid | C | A | C | C | C | C | C | C |
| ethanesulfonic acid | A | A | C | C | C | C | C | C |
| formic acid | A | A | A | A | A | A | C | C |
| fumaric acid | C | A | C | C | C | C | C | C |
| gluconic acid | A | A | A | A | A | A | A | A |
| hydrochloric acid | A | A | C | C | C | C | C | C |
| lactic acid | A | A | C | A | C | C | C | C |
| maleic acid | A | A | C | C | C | C | C | C |
| L-malic acid | A | A | A | A | C | C | C | C |
| DL-mandelic acid | A | A | C | C | C | C | C | C |
| methanesulfonic acid | A | A | A | C | C | C | C | C |
| naphthalene-1,5-disulfonic | C | C | C | C | C | C | C | C |
| acid | | | | | | | | |
| naphthalene-2-sulfonic acid | A | A | C | A | C | A | C | C |
| nitric acid | A | A | C | C | C | C | C | C |
| octadecanoic acid | A | A | C | A | C | C | C | C |
| p-toluenesulfonic acid | A | A | C | C | C | C | C | C |
| phosphoric acid | C | P | C | C | C | C | C | C |
| propanedioic acid | A | A | C | C | A | C | C | C |
| succinic acid | A | A | C | C | A | C | C | C |
| sulfuric acid | A | A | A | C | C | C | C | C |
| L-tartaric acid | A | A | C | C | B | B | C | B |
| undec-10-enoic acid | A | A | A | A | A | A | A | A |
| p-xylene-2-sulfonic acid | C | A | C | C | C | A | C | C |

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A salt of 11-piperazin-1-yldibenzo[b,f][1,4]thiazepine, wherein said salt is:
a) an L-tartaric acid salt;
b) a fumaric acid salt;
c) a methanesulfonic acid salt;
d) a sulfuric acid salt;
e) a phosphoric acid salt;
f) an acetic acid salt;
g) an L-ascorbic acid salt;
h) a benzoic acid salt;
i) a benzene sulfonic acid salt;
j) a 2-(benzoylamino)acetic acid salt;
k) a (+)-(1S)-camphor-10-sulfonic acid salt;
l) a citric acid salt;
m) a 1,2-ethanedisulfonic acid salt;
n) an ethanesulfonic acid salt;
o) a formic acid salt;
p) a gluconic acid salt;
q) a lactic acid salt;
r) a maleic acid salt;
s) an L-malic acid salt;
t) a DL-mandelic acid salt;
u) a naphthalene-1,5-disulfonic acid salt;
v) a naphthalene-2-sulfonic acid salt;
w) a nitric acid salt;
x) an octadecanoic acid salt;
y) a p-toluenesulfonic acid salt;
z) a propanedioic acid salt;
aa) a succinic acid salt;
bb) an undec-10-enoic acid salt; or
cc) a p-xyelene-2-sulfonic acid salt.

2. The salt of claim 1 wherein said salt is crystalline.

3. A composition comprising a salt of claim 1.

4. The composition of claim 3 further comprising a pharmaceutically acceptable carrier.

5. The composition of claim 4 further comprising at least one benzodiazepine, 5-HT_{1A} ligand, 5-HT_{1B} ligand, 5-HT_{1D} ligand, mGluR2A agonist, mGluR5 antagonist, antipsychotic, NK1 receptor antagonist, antidepressant, or serotonin reuptake inhibitor.

6. A process for preparing any one of the salts of claim 1 comprising combining 11-piperazin-1-yldibenzo[b,f][1,4]thiazepine together with:
a) L-tartaric acid;
b) fumaric acid;
c) methanesulfonic acid;
d) sulfuric acid,
e) phosphoric acid;
f) acetic acid;
g) L-ascorbic acid;
h) benzoic acid;
i) benzene sulfonic acid
j) 2-(benzoylamino)acetic acid;
k) (+)-(1S)-camphor-10-sulfonic acid;
l) citric acid;
m) 1,2-ethanedisulfonic acid;
n) ethanesulfonic acid;
o) formic acid;
p) gluconic acid;
q) lactic acid;
r) maleic acid;
s) L-malic acid;
t) DL-mandelic acid;
u) naphthalene-1,5-disulfonic acid;
v) naphthalene-2-sulfonic acid;
w) nitric acid;
x) octadecanoic acid;
y) p-toluenesulfonic acid;
z) propanedioic acid;
aa) succinic acid;
bb) undec-10-enoic acid; or
cc) p-xyelene-2-sulfonic acid
in a solvent and precipitating said salt from said solvent.

7. The process of claim 6 wherein said 11-piperazin-1-yldibenzo[b,f][1,4]thiazepine and acid are combined in a molar ratio of about 5:1 to about 1:5.

8. The process of claim 6 wherein said 11-piperazin-1-yldibenzo[b,f][1,4]thiazepine and acid are combined in a molar ratio of about 1:1 to about 1:2.

9. The process of claim 6 wherein said solvent comprises acetonitrile, methanol, acetone, ethyl acetate, or mixture thereof.

10. A salt of 11-piperazin-1-yldibenzo[b,f][1,4]thiazepine prepared by the process of claim 6.
